(19) United States
(12) Patent Application Publication
(10) Pub. No.: US 2025/0121145 A1
(43) Pub. Date: Apr. 17, 2025

(54) ARRANGEMENT AND PROCESS FOR MEASURING THE RESPECTIVE CONCENTRATION OF THREE GAS COMPONENTS IN A GAS SAMPLE

(71) Applicant: Drägerwerk AG & Co. KGaA, Lübeck (DE)

(72) Inventors: Hartmut STARK, Lübeck (DE); Hans-Ullrich HANSMANN, Lübeck (DE); Bernd-Michael DICKS, Lübeck (DE); Robert JAHNS, Lübeck (DE); Hendrik KÄHLER, Lübeck (DE)

(21) Appl. No.: 18/911,559

(22) Filed: Oct. 10, 2024

(30) Foreign Application Priority Data
Oct. 13, 2023 (DE) 10 2023 128 115.1

(51) Int. Cl. A61M 16/00; A61M 16/08; A61M 16/10; A61M 16/12; G01N 1/44; G01N 9/00; G01N 25/18; G01N 33/00

(52) U.S. Cl. CPC A61M 16/022; A61M 16/085; A61M 16/1005; A61M 16/12; G01N 1/44; G01N 9/002; G01N 25/18; G01N 33/0027; A61M 2016/1025; A61M 2016/1035; A61M 2205/3327; A61M 2205/3368

(57) ABSTRACT

A sensor arrangement and a measuring process measure the respective concentration of three gas components of a gas sample (Gp). One gas component is a paramagnetic gas. The gas sample (Gp) is fed into a measuring chamber (2). A magnetic field with an oscillating magnetic field strength is applied to the measuring chamber (2). The thermal conductivity of the gas sample (Gp) and the magnetically modulated thermal conductivity of the gas sample in the measuring chamber (2) are measured. The three concentrations of the three gas components are determined using the predetermined densities of the three gas components as well as the measured thermal conductivity, the measured magnetically modulated thermal conductivity and the measured density.

### CROSS REFERENCE TO RELATED APPLICATIONS

This application claims the benefit of priority under 35 U.S.C. § 119 of German Application 10 2023 128 115.1, filed Oct. 13, 2023, the entire contents of which are incorporated herein by reference.

### TECHNICAL FIELD

The invention relates to a sensor arrangement and a measuring process for measuring the respective concentration of three gas components of a gas sample.

### BACKGROUND

The task of measuring the respective concentration of several gas components in a gas sample occurs, for example, during artificial ventilation (artificial respiration) of a patient. A ventilator delivers a breathable gas mixture to a patient-side coupling unit, for example to a breathing mask or a tube, wherein the patient-side coupling unit is connected to the patient. The breathable gas mixture contains oxygen and, in one arrangement at least one anesthetic agent (anesthetic gas).

A process and device for measuring the oxygen concentration and the anesthetic concentration is described in US 2023 0114548 A1. A magnetic field with an oscillating magnetic field strength is generated in a measuring chamber. The thermal conductivity and the magnetically modulated thermal conductivity of the breathable gas mixture are measured. Use is made of the fact that oxygen is a paramagnetic gas.

### SUMMARY

It is an object of the invention to provide a sensor arrangement and a measuring process which are capable of measuring the respective concentration of three gas constituents of a gas sample, namely with a higher reliability than known sensor arrangements and measuring processes.

The problem is solved by a sensor arrangement with features according to the invention and by a measuring process with features according to the invention. Advantageous embodiments of the sensor arrangement according to the invention are, where appropriate, also advantageous embodiments of the measuring process according to the invention and vice versa.

The sensor arrangement according to the invention and the measuring process according to the invention are capable of measuring the respective concentration (the respective proportion/share) of three different gas components of a gas sample. Preferably, “the concentration” is understood to mean the respective proportion in % by volume (vol.-%), the concentration can also be the proportion in % by weight, i.e. the quantity share. One gas component of the gas sample is a paramagnetic gas, in particular oxygen.

Remark: A paramagnetic gas has the following properties: The atoms or molecules of the gas comprises permanent magnetic properties, i.e. a magnetic moment, and automatically align in a magnetic field.

The respective density and the respective thermal conductivity of each one of the three gas components are predetermined in a computer-evaluable form. For example, the sensor arrangement has at least temporary read access to a data memory in which the densities and thermal conductivities are stored.

The sensor arrangement comprises a measuring chamber, a thermal conductivity sensor and a density sensor. The measuring chamber is configured to accommodate a gas sample. The thermal conductivity sensor is configured to measure the thermal conductivity of the gas sample in the measuring chamber. The density sensor is configured to measure the density of the gas sample. The measuring process according to the invention is carried out using such a sensor arrangement and comprises the steps of measuring the thermal conductivity and the density of the gas sample.

Note: The formulation that a sensor measures a physical quantity means the following: The sensor directly measures the physical quantity or at least one other physical quantity, wherein the or each other physical quantity correlates with the physical quantity to be measured. The or each other physical quantity is therefore an indicator of the physical quantity to be measured.

The measuring process according to the invention further comprises the following steps, and the sensor arrangement according to the invention is configured to perform the following steps:
The gas sample is fed or guided into the measuring chamber.
A magnetic field with an oscillating magnetic field strength is applied to the measuring chamber.
The magnetically modulated thermal conductivity of the gas sample in the measuring chamber is measured.

The “magnetically modulated thermal conductivity” of the gas sample is understood to be the proportion (share) of the thermal conductivity that oscillates depending on the magnetic field strength. This magnetically modulated thermal conductivity depends essentially, but usually not exclusively, on the concentration of the paramagnetic gas in the gas sample. It is possible that the thermal conductivity measured as an indicator of the (total) thermal conductivity of the gas sample is the thermal conductivity of the gas sample in the measuring chamber. It is possible that the same thermal conductivity sensor measures both the thermal conductivity and the magnetically modulated thermal conductivity, i.e. provides two different signals that can be independent of each other. It is also possible that two different thermal conductivity sensors are used.

The three gas component concentrations are determined using the following information:
the predetermined densities of the three gas components,
the predetermined thermal conductivities of the three gas components,
the measured thermal conductivity of the gas sample,
the measured magnetically modulated thermal conductivity of the gas sample, and
the measured density of the gas sample,
optionally additionally the temperature of the gas sample,
optionally additionally the temperature in the measuring chamber and
optionally additionally the pressure of the gas sample, preferably the pressure in the measuring chamber.

According to the invention, three different properties of the gas sample are measured, namely the thermal conductivity, the magnetically modulated thermal conductivity and the density. As a rule, these three properties of the gas sample depend on the sought concentrations of the three gas components in the gas sample, but do not depend on each other. In particular, these three properties generally change independently of each other. With the help of these three independent properties of the gas sample, which properties have been determined by measurements and are therefore known, three unknowns can be determined, namely the three concentrations of the three gas components sought.

The invention does not require the gas sample to consist only of these three gas components. Rather, the gas sample can comprise at least one further gas component. In order to determine its concentration, in many cases the fact that the sum of the concentrations of the gas components, measured in vol.-% or in wt.-%, is 100% can be utilized.

In some applications, in particular the concentration of the paramagnetic gas is required, wherein the paramagnetic gas is oxygen in particular. Some known sensor arrangements and measuring processes are also capable of measuring the oxygen concentration in a gas sample, but have a relatively high cross-sensitivity to other components of the gas sample. Such a cross-sensitivity may lead to wrong measurement results and is therefore undesirable. The sensor arrangement according to the invention and the measuring process according to the invention, on the other hand, are significantly less cross-sensitive, ideally not cross-sensitive at all, to other gas components in the gas sample.

Thanks to the invention, in many cases it is not necessary to provide a reference gas with a known composition and use it for the measurement, while the sensor arrangement and the measurement process measure the gas component concentrations.

Electrochemical sensors have become well known. In an electrochemical sensor, a chemical process takes place that depends on the concentration of oxygen and/or another gas component in the gas sample. A detection variable sensor of the electrochemical sensor measures an electrical detection variable that correlates with the oxygen concentration being sought. In some cases, such electrochemical sensors have the disadvantage that they require a chemical that is consumed over time and therefore needs to be replenished from time to time. In addition, a chemical can be altered by environmental influences, in particular by ambient heat, or can evaporate. The invention does not require a chemical and therefore avoids these disadvantages.

In one application, the paramagnetic gas component is oxygen. In this application, the two other gas components, whose concentrations are measured according to the invention, are an anesthetic agent (an anesthetic gas) or carbon dioxide and argon. The noble gas argon occurs in the ambient air at a known concentration. However, the concentration of argon in the gas sample is in many cases higher than the known concentration of argon in the ambient air. This situation occurs in particular in the following application: The gas sample comes from a breathable gas mixture that is used to artificially ventilate a patient. The gas mixture is produced using ambient air, but has a higher oxygen content than ambient air. A so-called concentrator is used to produce the gas mixture from ambient air. If a gas mixture with a higher oxygen content than ambient air is produced using a concentrator, the gas mixture produced usually also has a higher concentration of argon than the ambient air. This higher concentration is measured in the application of the invention just described.

Preferably, the sensor arrangement is capable of simultaneously measuring both the thermal conductivity and the magnetically modulated thermal conductivity of a gas sample in the same measuring chamber. In one embodiment, the sensor arrangement comprises a heating element and is capable of heating the heating element, in particular by applying an electrical voltage to the heating element and causing an electrical current to flow through an electrical heating wire of the heating element. When the heating element is heated, it supplies thermal energy to a gas sample in the measuring chamber. Preferably, this heating element does not react chemically with the gas sample in the measuring chamber.

According to the invention, a magnetic field with an oscillating magnetic field strength is applied to the measuring chamber. The thermal conductivity and the magnetically modulated thermal conductivity of the gas sample are measured in the measuring chamber. Preferably, an electrical detection variable is measured. This electrical detection variable correlates with the thermal conductivity of the gas sample in the measuring chamber. Electrical or electronic filtering of the electrical detection variable is carried out. This filtering provides two signals, namely an oscillating signal and another signal. The oscillating signal oscillates depending on the oscillating magnetic field strength. The time course of the other signal ideally does not depend on the oscillating magnetic field strength. The oscillating signal is used as the or as an indicator of the magnetically modulated thermal conductivity. The oscillating signal thus correlates with the concentration of the paramagnetic gas in the gas sample. The other signal is used as the or an indicator of the thermal conductivity of the gas sample. It correlates with the thermal conductivity of the gas sample.

In many cases, this design results in a particularly compact sensor arrangement. Two independent properties of the gas sample are measured in the same measuring chamber using the same magnetic field generator and the same heated heating element, namely the thermal conductivity and the magnetically modulated thermal conductivity.

Various configurations are possible as to how the density of the gas sample is measured. In one embodiment, a mechanical or electronic flexural resonator (flexural oscillator) is used. This flexural resonator comprises an oscillating body. In the case of a mechanical flexural resonator, this resonator is or comprises a movably mounted fluid guide unit. A “fluid guide unit” is a component that guides a fluid along a trajectory, wherein this trajectory is predetermined by the configuration and arrangement of the component. A corrugated hose, a smooth hose and a tube are examples of a fluid guide unit. The fluid guide unit does not necessarily include a conveying unit. In the case of an electronic flexural resonator, this oscillating body is preferably a quartz crystal.

The gas sample is passed through the fluid guide unit of the flexural resonator or past the quartz crystal. The flexural resonator is configured to cause the oscillating body (fluid guide unit or quartz crystal) to oscillate while a part of the gas sample is in the fluid guide unit or is guided past the oscillating body or surrounds the oscillating body. The natural frequency of the oscillating body is measured. This measured natural frequency correlates with the mass of the gas sample.

In the case of a mechanical flexural resonator, the natural frequency correlates with the mass of the filled fluid guide unit. As a rule, this mass is the sum of the mass of the empty fluid guide unit and the sought mass of the gas sample in the fluid guide unit. The mass of the empty fluid guide unit is known by the configuration of the flexural resonator and does not change. The volume of the fluid guide unit is also known from the configuration and remains constant during a measurement. The sought density of the gas sample can be derived from the measured natural frequency and the mass and volume of the empty fluid guide unit.

The principle of such a flexural resonator has been known for a long time, and reliable flexural resonators for density measurement are available.

In one embodiment, the concentration of at least a fourth gas component of the gas sample is measured directly. The respective concentration of the or each fourth gas component of the gas sample is used for the step of determining the respective concentration of the three gas components. In many cases, this embodiment increases the reliability with which the three concentrations of the three gas components are determined.

In one implementation of this embodiment, the concentration of the or at least a fourth gas component is measured by a photo-electric sensor using the following well-known principle:
A radiation source emits electromagnetic radiation or sound or ultrasound into a measuring section or measuring chamber and emits it with sufficient broadband. The gas sample flows through this measuring section or is located in this measuring chamber. The measuring chamber can be the measuring chamber with the magnetic field strength just described or another measuring section or measuring chamber being remotely arranged.
The electromagnetic radiation or sound penetrates the measuring section or the measuring chamber. At least a part impinges on a detector.
The gas sample in the measuring section or measuring chamber absorbs part of the radiation or sound. This reduces the intensity of the radiation or sound in a wavelength range.
The detector measures the intensity of the impinging radiation or the impinging sound. This intensity correlates with the sought concentration of the fourth gas component.
The detector generates a signal depending on the measured intensity. This signal contains information about the intensity and therefore also about the concentration of the fourth gas component.

Many gases exhibit a characteristic spectral course of absorption, i.e. a characteristic dependence of the degree of absorption on the wavelength of the electromagnetic radiation or sound that penetrates this gas. As a rule, the concentration of the fourth gas component can be measured independently of the sought concentrations of the three gas components.

According to the invention, three properties of the gas sample are measured in order to determine values for three unknowns, the three unknowns being the three sought concentrations of the three gas components in the gas sample. In one embodiment, three functional relationships, three difference indicators and a target function (objective function) are predetermined. Preferably, the sensor arrangement according to the invention has at least temporary read access to a data memory in which the target function, the difference indicators and the functional relationships are stored in a form that can be analyzed by a computer.

The first functional relationship describes the thermal conductivity of the gas sample as a function of the three gas component concentrations sought. The second functional relationship describes the magnetically modulated thermal conductivity of the gas sample as a function of the three gas component concentrations. The third functional relationship describes the density of the gas sample as a function of the three gas component concentrations. Therefore, all three functional relationships depend on the three unknown and sought gas component concentrations.

The first difference indicator describes an indicator for a difference between the measured thermal conductivity of the gas sample and the thermal conductivity, which is derived using the first functional relationship. The second difference indicator describes an indicator for a difference between the measured magnetically modulated thermal conductivity of the gas sample and the magnetically modulated thermal conductivity, which is derived with the aid of the second functional relationship. The third difference indicator describes an indicator of a difference between the measured density of the gas sample and the density derived using the third functional relationship. Preferably, each difference indicator is the square of the respective difference, alternatively the magnitude of the difference or another function which is the greater the greater the magnitude of the difference.

The target function is an aggregation of these three difference indicators. Preferably, the target function is a weighted average of the three difference indicators. For example, the weighting factors compensate for different orders of magnitude of the three properties thermal conductivity, magnetically modulated thermal conductivity and density. It is also possible that additionally or in lieu the weighting factors take into account different accuracy requirements for the gas component concentrations sought.

The three measured values for the gas sample, i.e. the measured thermal conductivity, the magnetically modulated thermal conductivity and the density of the gas sample, are inserted into the target function. After insertion, the target function depends on three unknowns, namely the three gas component concentrations sought. The target function is numerically minimized. This means that different value triplets for the three gas component concentrations are inserted into the target function on a trial basis. Each value triplet assigns a value to each of the three concentrations. The value triplet or a value triplet that leads to a minimum value of the target function provides the three gas component concentrations sought. Ideally, the minimizing value triplet provides the value zero for the target function. In practice, there is always a gap (difference) between the measured property and the property predicted according to the functional relationship. Preferably an iterative minimization procedure is applied.

It is possible that the target function has several minima.

It is possible that the concentration of a fourth gas component also occurs in at least one functional relationship. In one implementation, a sensor directly measures the concentration of this fourth gas component, for example using the photoelectric measuring principle described above, comprising a radiation source and a detector. In another implementation, a standard value is predetermined for the concentration of the fourth gas component. It is possible that there is a further, i.e. a fifth gas component whose concentration is measured or for which a standard value is predetermined.

The invention further relates to a ventilation arrangement (ventilator system) and a ventilation process for artificially ventilating a patient. The ventilation arrangement comprises
a medical device, in particular a ventilator,
a patient-side coupling unit,
a ventilation control unit and
a sensor arrangement according to the invention.

The ventilation process (procedure) is carried out using such a ventilation arrangement.

The patient is connected to the patient-side coupling unit or can at least temporarily be connected to a patient-side coupling unit. In particular, the patient-side coupling unit comprises a breathing mask and/or a tube and/or a catheter. The ventilation arrangement is capable of conducting and/or conveying a breathable gas mixture from the medical device to the patient-side coupling unit. The gas mixture contains oxygen and, in one embodiment, at least one anesthetic and/or a drug. It is possible that a ventilation circuit is established. The gas mixture exhaled by the patient flows in the ventilation circuit back to the medical device.

The concentration of at least one gas component of the breathable gas mixture, in particular the concentration of oxygen, should lie within a predetermined target range. It is possible that this target range is variable over time. It is also possible that a respective target range is predetermined for at least two gas components.

The ventilation arrangement is configured to perform the following steps, and the ventilation process comprises the following steps:
A gas sample is branched-off from the gas mixture that is conveyed to the patient-side coupling unit.
The branched-off gas sample is passed to the sensor arrangement.
The sensor arrangement measures at least the concentration of the or each gas component, for which a target range is predetermined, in the branched-off gas sample, optionally the concentration of at least one other gas component.

The ventilation control unit performs closed-loop control or open-loop control. The controlled variable is the actual concentration of at least one gas component in the gas mixture that is fed to the patient-side coupling unit. The control target (control gain) for the closed-loop control is that the control variable, i.e. the actual gas component concentration, is within the predetermined target range. For the closed-loop control, the ventilation control unit uses the gas component concentration in the branched-off gas sample, wherein the sensor arrangement has measured this concentration.

The invention is described below by means of embodiment examples. The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

In the drawings:

FIG. 1 is a schematic view of a ventilation circuit for artificial ventilation of a patient;

FIG. 2 is a schematic view of a first embodiment of a sensor capable of measuring the concentration of oxygen and another gas;

FIG. 3 is a schematic view of a second embodiment of such a sensor;

FIG. 4 is a schematic view of a measuring unit of the sensor in FIG. 3;

FIG. 5 is a schematic view of an exemplary evaluation circuit of the sensor shown in FIG. 2 and FIG. 3;

FIG. 6 is a schematic view of a mechanical flexural resonator with which the density of the gas sample is measured;

FIG. 7A is a perspective view of an electronic flexural resonator in the form of an oscillating quartz crystal, which is used to measure the density of the gas sample;

FIG. 7B is a schematic view of an electronic flexural resonator illustrating oscillations of two legs;

FIG. 8 is a graph illustrating the magnetically modulated thermal conductivity λ2f of a gas mixture as a function of the oxygen concentration.

### DESCRIPTION OF PREFERRED EMBODIMENTS

Referring to the drawings, in the embodiment examples, the sensor arrangement according to the invention is used for the artificial ventilation of a patient. The patient is supplied with a breathable gas mixture comprising oxygen and at least one anesthetic agent (anesthetic). The patient is therefore anaesthetized or at least sedated.

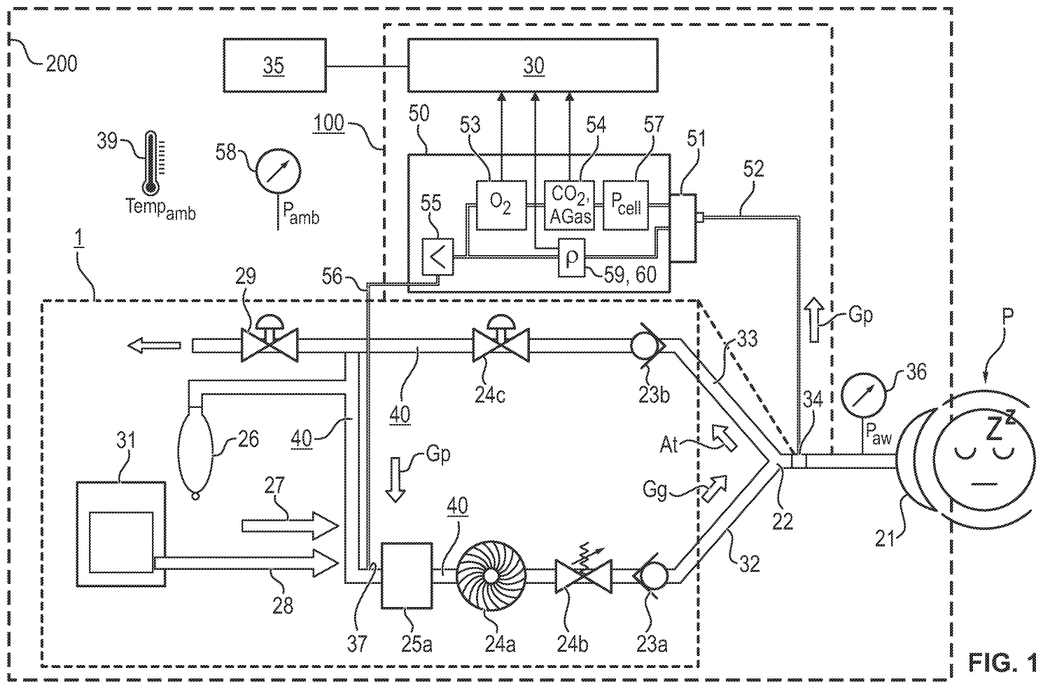

FIG. 1 schematically shows a ventilation arrangement 200 with a schematically shown ventilator 1, wherein the ventilation arrangement 200 is configured to artificially ventilate a patient P. Only the face of the patient P is shown schematically. In the example shown, the ventilator 1 maintains a ventilation circuit 40. In the example shown, the ventilator 1 is configured as an anesthetic device, and the patient P is anaesthetized or at least sedated.

In the ventilation circuit 40, a breathable gas mixture Gg is supplied to the patient P. In the embodiment example, this gas mixture Gg comprises oxygen (O₂), at least one anesthetic agent and optionally a carrier gas for an anesthetic agent, e.g. nitrous oxide (N₂O) and optionally also a drug. An anesthetic dispenser 31 generates a fluid flow 28 with vaporous anesthetic and a carrier gas. The fluid flow 28 is fed into the ventilation circuit 40. In addition, a fluid flow 27 of fresh air or other fresh gas is fed in at least intermittently. At least as long as there is no excessive overpressure in the ventilation circuit 40, the gas mixture exhaled by the patient P remains in the ventilation circuit 40 so that no anesthetic agent enters the environment.

The gas mixture At, which is exhaled by the patient P and flows through the ventilation circuit 40, inevitably contains carbon dioxide (CO₂). A carbon dioxide absorber (CO₂ absorber) 25a is configured to absorb carbon dioxide from the ventilation circuit 40. In addition, the breathable gas mixture Gg may contain other components that also occur in the ambient air, albeit possibly at a different concentration than in ambient air.

The invention can also be used in an application in which no ventilation circuit is implemented. The gas mixture At exhaled by the patient P passes through an expiratory line 33 into the environment.

A patient-side coupling unit 21, shown only schematically, for example a mouthpiece or a breathing mask or a tubus, connects the patient P to the ventilation circuit 40. A tube close to the patient connects the patient-side coupling unit 21 to the base part of a Y-piece 22. One limb of the Y-piece 22 is connected to an inspiration line 32 for inhalation (inspiration), the other limb to an expiration line 33 for exhalation (expiration).

A fluid delivery unit 24a sucks in gas and generates a permanent flow of breathable gas mixture Gg through the inspiration line 32 to the Y-piece 22 and to the patient-side coupling unit 21 and thus to the patient P. The ventilation circuit 40 is kept going by the fluid delivery unit 24a and optionally by a breathing bag 26, which can be operated manually. The fluid delivery unit 24a can be implemented in particular as a blower, a pump or a piston-cylinder unit.

A non-return valve (check valve) 23a allows a gas flow through the inspiration line 32 towards the Y-piece 22 and blocks a gas flow in the opposite direction. A non-return valve 23b allows a gas flow to pass through the expiration line 33 away from the Y-piece 22 and blocks a gas flow in the opposite direction.

In the embodiment example, the fluid conveying unit 24a continuously conveys the gas mixture Gg. A controllable valve 24b, preferably a proportional valve, allows the flow of the gas mixture generated by the fluid conveying unit 24a to pass or blocks it completely or entirely, depending on its position, thereby contributing to the generation of the individual ventilation strokes and determining the amplitudes and frequencies of these ventilation strokes.

Additionally, a PEEP valve 24c (PEEP=“positive end-expiratory pressure”) ensures that a sufficiently high air pressure is maintained in the lungs of patient P even at the end of exhalation or if the ventilation circuit 40 is briefly opened or interrupted. This reduces the risk of patient P's lungs collapsing due to insufficient pressure.

A pressure relief valve 29 is configured to reduce excess pressure in the ventilation circuit 40 by causing gas to escape from the ventilation circuit 40 if the pressure is too high. Preferably, the released gas is discharged into a transport line for anesthetic gas and reaches a conditioner not shown.

A signal-processing ventilation control unit 35, shown only schematically, receives a signal from an optional pressure sensor 58, which measures the air pressure P_{amb} in the environment of the anesthesia machine 1 and thus of the ventilation circuit 40, and a signal from an optional temperature sensor 39, which measures the ambient temperature Temp_{amb}. In addition, the ventilation control unit 35 receives measured values from a pressure sensor 36, which measures the current pressure in the ventilation circuit 40, for example the ventilation pressure applied to the patient P (pressure in airway, Paw), in one embodiment as a differential pressure relative to the ambient pressure P_{amb}. The measuring position of the pressure sensor 36 can be in the inspiration line 32 or in or, as shown, on or near the Y-piece 22. Preferably, the measuring position of the pressure sensor 36 is located on the tube close to the patient. The ventilation control unit 35 controls the fluid delivery unit 24a, the valve 24b, the anesthetic dispenser 31 and other components of the ventilation circuit 40 in order to realize a desired artificial ventilation and anesthesia of the patient P.

It is often desired that the gas mixture Gg, which is supplied to the patient P, fulfills a certain given property. In particular, the concentration of a component of the gas mixture Gg should be within a predetermined target range. In particular, the proportion of pure oxygen (O₂) should often be within a predetermined target range, for example between 40 vol.-% and 50 vol.-% or between 25 vol.-% and 30 vol.-%. Or the proportion of anesthetic should be within a predetermined range. In the following, the term “concentration” of a gas component in a gas mixture is used. Synonymous terms are “proportion” or “share”.

For this purpose, a sample of gas (hereinafter: a gas sample Gp) is branched off (diverted) from the gas mixture Gg, At in the ventilation circuit 40 via a gas sample fluid guide unit in the form of a sampling tube 52, analyzed and fed back into the ventilation circuit 40 via a discharge tube 56. The sampling tube 52 starts at a branching point 34 between the patient-side coupling unit 21 and the Y-piece 22. Because the branching point 34 is positioned at this point, the gas sample Gp contains a sample from the breathable gas mixture Gg or from the exhaled gas mixture At, depending on the time of branching-off. At times a gas mixture Gg from the inspiration line 22 and at times a gas mixture At for the expiration line 33 enter the sampling tube 52. At the branch point 34 there is an optional valve, not shown, which in a closed position separates the sampling tube 52 from the ventilation circuit 40 and which can be controlled by the ventilation control unit 35. When the valve is fully open or left open or omitted, the sampling tube 52 is in an unrestricted fluid connection with the ventilation circuit 40. The discharge tube 56 leads to a confluence point 37 upstream of the carbon dioxide absorber 25a.

The sampling tube 52 directs the gas sample Gp to a sensor arrangement 50. This sensor arrangement 50 is spatially remote from the patient-side coupling unit 21 and from the lines 32 and 33 and preferably belongs to the schematically shown ventilator 1. In FIG. 1, the sensor arrangement 50 is shown outside the ventilator 1 for clarity.

In the embodiment example, the sensor arrangement 50 comprises a pump 55, which sucks the gas sample Gp out of the ventilation circuit 40 and sucks it in through the sampling tube 52. Preferably, the pump 55 generates a permanent negative pressure on the side pointing towards the sampling tube 52 and a permanent positive pressure on the side pointing towards the discharge tube 56. The pump 55 is capable of generating a volume flow which is preferably constant over time and amounts to 200 ml/min, for example. A water trap 51 is arranged at the inlet of the sensor arrangement 50, wherein moisture is condensed and collected in the water trap 51. This reduces the water content in the gas sample Gp.

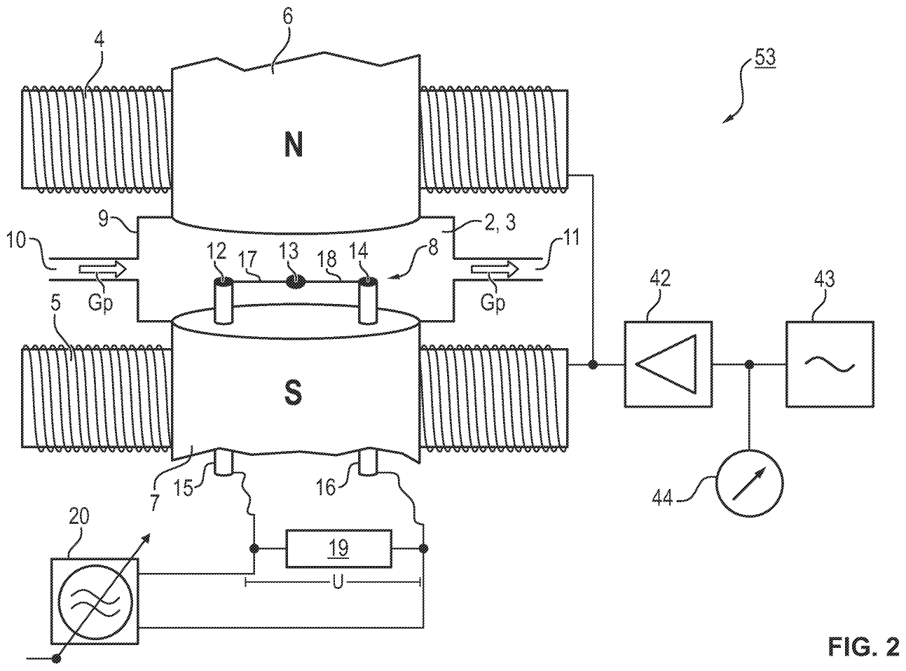

A sensor 54 is configured to generate signals, wherein the signals correlate with the measured respective concentration of CO₂ and of N₂O in the extracted gas sample Gp. Preferably, this sensor 54 comprises an infrared measuring head which utilizes the dipole moment of molecules in the gas sample Gp and quantitatively evaluates the absorption of infrared-active gases in order to determine the respective concentration. Preferably, the sensor 54 comprises a radiation source that emits electromagnetic radiation and a detector that measures the intensity of impinging electromagnetic radiation and generates a corresponding signal. A sensor 53 is configured to generate a signal that correlates, among other things, with the concentration of O₂ or another paramagnetic component of the gas sample Gp. The sensor 53 is described in more detail below with reference to FIG. 2 to FIG. 5.

In addition, the sensor arrangement 50 comprises a pressure sensor 57, which measures the pressure P_{cell} of the gas sample Gp at the inlet of the sensor arrangement 50. This pressure P_{cell} is variable over time because the pressure in the ventilation circuit 40 oscillates and because the sampling tube 52 is in a fluid connection with the ventilation circuit 40 if there is no valve at the branch point 34 or as long as the optional valve at the branch point 34 is open. If the valve is missing or open, the pressure in the ventilation circuit 40 propagates at approximately the sound velocity to the sensor arrangement 50.

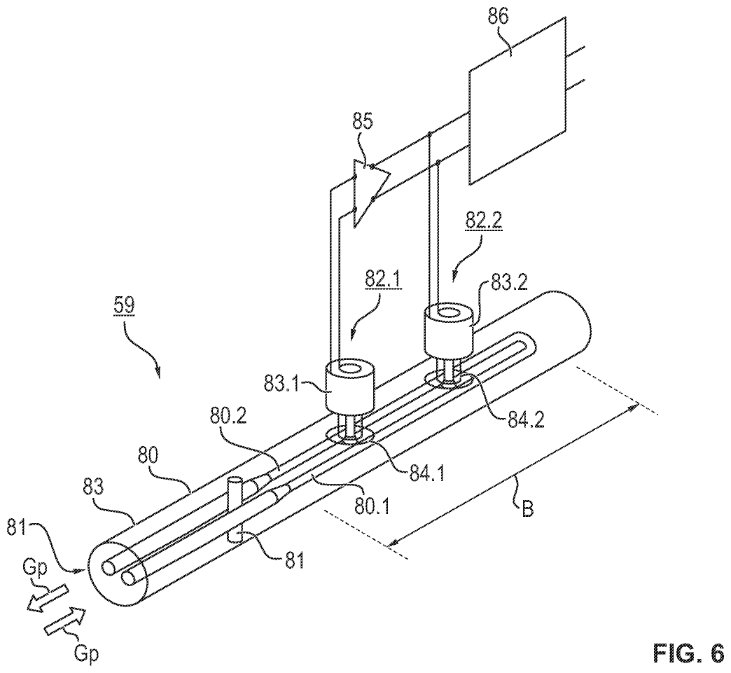

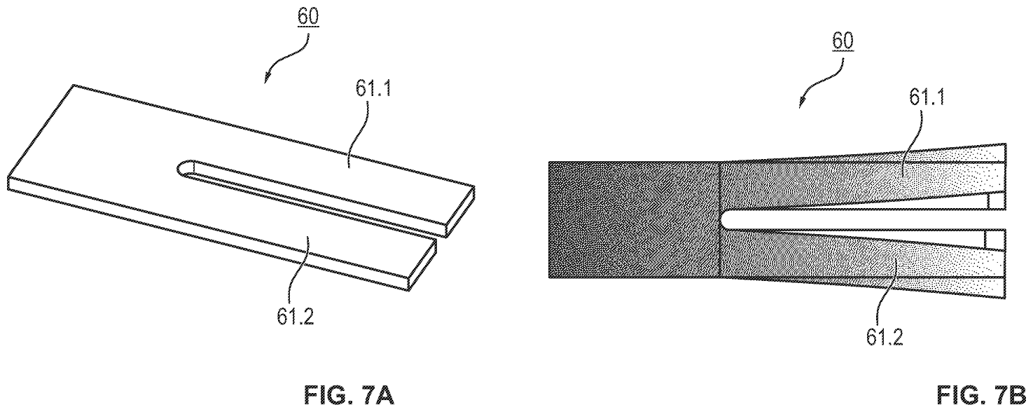

A density sensor 59, 60 measures the density of the gas sample Gp flowing through the sensor arrangement 50. This density sensor 59, 60 is described below with reference to FIG. 6 and FIG. 7. In the example of FIG. 1, the density sensor 59, 60 is arranged parallel to the sensors 53, 54, 57. It is also possible that the sensors 53, 54, 57 are arranged in series with the density sensor 59, 60 and that the tapped gas sample Gp flows through all four sensors.

The sensor arrangement 50, the water trap 51, the signal processing unit 30, and the tubes 52 and 56 belong together to a measurement system 100, which sucks in the gas sample Gp from the ventilation circuit 40, examines (analyzes) it and feeds it back into the ventilation circuit 40. The measurement system 100 is part of the ventilation arrangement 200.

FIG. 2 shows an example of a sensor 53 that is configured to measure the respective concentration of oxygen (O₂) and another gas in the gas sample Gp. This sensor 53 utilizes the fact that oxygen is a paramagnetic gas, whereas any other gas that is or may be present in the gas sample Gp is not paramagnetic.

Two pole pieces (pole shoes) 6, 7 and two field coils 4, 5 generate a magnetic field. An air gap 3 occurs between the two pole pieces 6, 7, which acts as a measuring chamber 2. This measuring chamber 2 is bounded by the two pole pieces 6, 7 and a wall 9. An inlet 10 and an outlet 11 are recessed into the wall 9.

A thermocouple 8 is attached to two support wires 15, 16 at two connection points 12 and 14, wherein the two support wires 15, 16 are passed through the lower pole piece 7 and are in thermal contact with the lower pole piece 7. The thermocouple 8 comprises two wires 17, 18, which are connected to each other at a connection point 13. A voltage source 20 applies an alternating voltage to the two support wires 15, 16 and thus to the thermocouple 8. This heats the thermocouple 8 to a working temperature that is higher than the temperature of the gas sample Gp in the measuring chamber 2. An electrical voltage U, which is applied to a measuring resistor 19, is measured. This electrical voltage U contains an AC voltage component and a DC voltage component.

The current working temperature of the thermocouple 8 is measured at the connection point 13. This temperature depends on the one hand on the voltage U that occurs between the support wires 15, 16 and on the other hand on the thermal conductivity 2 of the gas sample Gp in the measuring chamber 2. Closed-loop control is performed with the control objective of keeping the working temperature of the thermocouple 8 to a constant value. The control variable is the time-varying voltage U, which the voltage source 20 applies to the support wires 15, 16. Because the temperature of the thermocouple 8 ideally remains constant, an electrical detection variable of the thermocouple 8 correlates with the thermal conductivity 2 of the gas sample Gp in the measuring chamber 2.

One way of measuring the detection variable is as follows: The time-varying electrical power supplied to the thermocouple 8 is measured as the detection variable. The two support wires 15 and 16 are electrically connected to each other by the electrical measuring resistor 19. The electrical voltage U that drops across this measuring resistor 19 is measured. The strength of the electric current flowing through the thermocouple 8 is also measured. The electrical power is known to depend on the voltage and the current. It is also possible to use the electrical voltage U as a detection variable.

A voltage source 43 is connected to one field coil 5 via a power amplifier 42, and the other field coil 4 is connected to electrical ground. The voltage source 43 applies a sinusoidal electrical voltage. This voltage generates a sinusoidally varying magnetic field in the measuring chamber 2. The thermocouple 8, which is heated to a constant temperature, emits a quantity of heat per time unit to the gas sample Gp in the measuring chamber 2. This amount of heat emitted per time unit correlates with the measured electrical power supplied to the thermocouple 8. Because the field strength of the magnetic field in the measuring chamber 2 oscillates periodically, the amount of heat emitted per time unit also oscillates-provided that a paramagnetic gas is present in the measuring chamber 2 as part of the gas sample Gp. The detection variable correlates with the thermal conductivity 2 of the gas sample Gp. Using the detection variable, a signal that oscillates depending on the oscillation of the magnetic field, for example an AC voltage, and a non-oscillating or less oscillating signal, for example a DC voltage, are generated. The oscillating signal correlates with the thermal conductivity of the paramagnetic part of the gas sample Gp and thus with the O₂ concentration. This thermal conductivity is also referred to as the magnetically modulated thermal conductivity 22f. The other signal correlates with the (total) thermal conductivity 2 of the entire gas sample Gp.

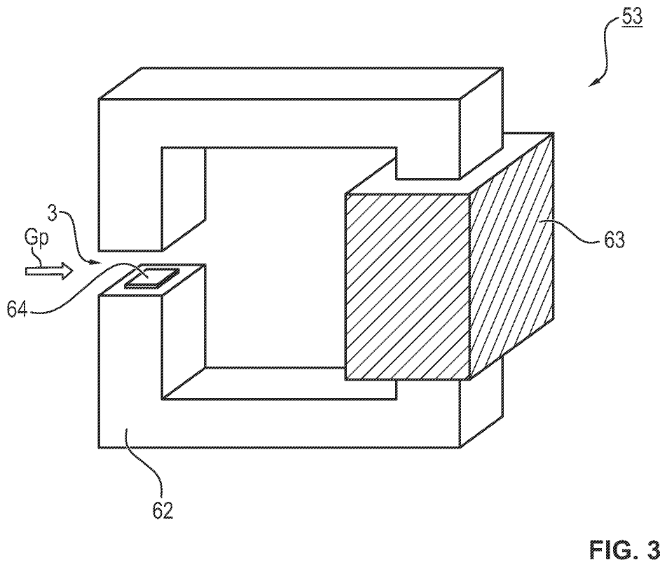

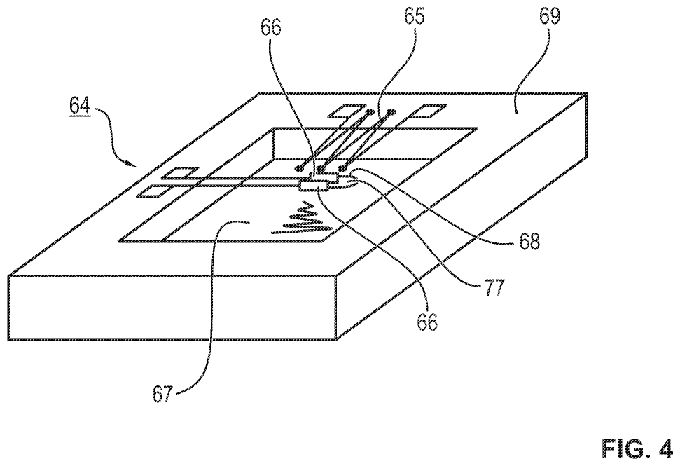

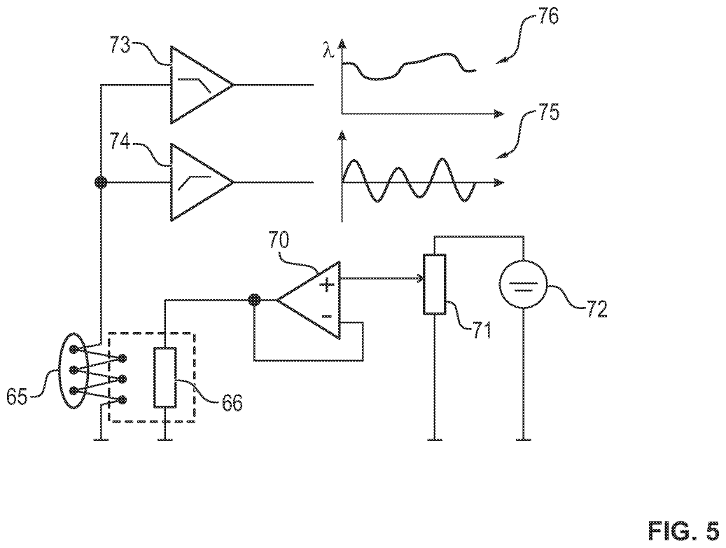

FIG. 3, FIG. 4 and FIG. 5 show a further embodiment of the sensor 53. The same reference signs have the same meanings as in FIG. 2. The circuit of FIG. 5 can also be used accordingly for the embodiment according to FIG. 2.

An electromagnet 62 with an electric coil 63 generates a time-varying, preferably oscillating, magnetic field in the air gap 3, see FIG. 3. The gas sample Gp to be examined reaches this air gap 3. The time characteristic of the strength of the generated magnetic field is determined by the control of the coil 63. Preferably, the strength of the magnetic field has a sinusoidal curve. A measuring unit 64 is arranged in the air gap 3, which is implemented as a chip, preferably as a semiconductor chip, which chip is manufactured using doped silicon, for example.

FIG. 4 shows the measuring unit 64 in detail. The measuring unit 64 comprises an electrically controllable heat conduction measuring element 65, an electrically controllable heating unit 66, and a diaphragm 67 in a frame 69. A gas sample Gp can pass through holes in the diaphragm 67 or around the diaphragm 67 to the elements 65, 66. The heating unit 66 can be an electrically conductive resistance structure deposited on the membrane 67 or can be configured as a heating wire. Two heating elements of the heating unit 66 are shown, which are electrically connected to each other by a connecting element 68. The heat conduction measuring element 65 and the heating unit 66 can also be configured as a one-piece element which has a temperature-dependent electrical resistance.

The heating unit 66 heats the measuring element 65 to a working temperature that is higher than the temperature of a gas sample Gp in the measuring chamber 2. The heated measuring element 65 measures the temperature at the measuring position 77. In the implementation shown, the measuring element 65 measures a thermoelectric voltage and utilizes the Seebeck effect. The thermal conductivity of the gas sample Gp at the measuring position 77 changes synchronously with the time-varying magnetic field generated by the electromagnet 62—provided that the gas sample Gp at the measuring position 77 contains a sufficiently high concentration of a paramagnetic gas. A higher thermal conductivity means that the thermal energy is better dissipated. This in turn results in a lower temperature, which in turn results in a lower thermoelectric voltage.

FIG. 5 shows an example of an evaluation circuit with the following components:
an amplifier 70, which is connected and arranged as an impedance converter,
a voltage divider 71 with variable tap,
a DC voltage source 72,
a low-pass filter 73 and
a high-pass filter 74, which is connected in parallel to the low-pass filter 73.

The heating unit 66 is connected to the DC voltage source 72 via the amplifier 70 and the voltage divider 71. The output signal of the heat conduction measuring element 65 is routed via the low-pass filter 73 and the high-pass filter 74. A signal 75 is present at the output of the high-pass filter 74, which signal oscillates depending on the oscillation of the magnetic field. The signal 75 correlates with the magnetically modulated thermal conductivity λ2f and thus with the concentration of the paramagnetic gas, for example with the concentration of oxygen, in the gas sample Gp. A signal 76 is present at the output of the low-pass filter 73, which signal does not oscillate at all or at least not synchronously with the magnetic field oscillation. The signal 76 correlates with the thermal conductivity 2 of the entire gas sample Gp.

Because the magnetic field strength oscillates, the amount of heat supplied per time unit comprises a superposition of a proportion that remains constant over time and a proportion that fluctuates periodically. The periodically fluctuating component correlates with the thermal conductivity and thus with the concentration of oxygen in the measuring chamber 2. The constant component, i.e. the component that does not oscillate depending on the strength of the magnetic field, correlates with the thermal conductivity of the entire gas sample Gp in the measuring chamber 2. The thermal conductivity of the oxygen and the respective thermal conductivity of each other component of the gas sample Gp contribute to this thermal conductivity 2 of the gas sample Gp. Both components are measured. The sensor 53 therefore provides two signals:
a signal that oscillates depending on the magnetic field strength, in this case periodically fluctuating, which is an indicator of the concentration of oxygen or another paramagnetic gas in the measuring chamber 2 (periodically fluctuating component, alternating voltage), and
a signal for the thermal conductivity 2 of the gas sample Gp in the measuring chamber 2, wherein this signal does not oscillate or at least does not oscillate depending on the magnetic field strength (constant component, DC voltage).

Optionally, a measured value for the concentration of a component and/or for the thermal conductivity 2 is displayed on a display device 44, see FIG. 2.

According to the invention, the density of the gas sample Gp is also measured. FIG. 6 schematically shows an embodiment of a density sensor 59 in the form of a mechanical flexural resonator. FIGS. 7A and 7B show an embodiment of an electronic flexural resonator 60, which is implemented as a quartz crystal. In one embodiment, the density sensor 59, 60 is connected in parallel with the sensor 53, which measures the thermal conductivity and the magnetically modulated thermal conductivity and was described above with reference to FIG. 2 to FIG. 5. It is possible that the sensor 53 is implemented as a chip and the density sensor 60 is mounted on this chip and is supplied with electrical voltage by this chip.

The principle of density measurement described below has been known for a long time, but not for the application described here. In the embodiment example, the density sensor 59, 60 measures the density p of the gas sample Gp flowing through the sensor arrangement 50.

First, the mechanical flexural resonator 59 is described with reference to FIG. 6. The gas sample Gp, whose density p is to be measured, is passed through a U-shaped tube 80 with two legs 80.1, 80.2. The tube 80 is located in a housing 83 and is made of a material that can chemically withstand the components of the gas sample Gp, for example made of glass or steel. The two legs 80.1, 80.2 of the tube 80 form the spring elements of a flexural resonator. This principle is similar to the principle of a tuning fork. The two legs 80.1, 80.2 are clamped at a fastening point 81. An area B of the tube 80 can be set into vibrations. This area B also has the shape of a U, comprises one segment of each leg 80.1, 80.2 and is bounded by the attachment point 81.

In one embodiment, the tube 80 can be temporarily closed for the purpose of a measurement. In another embodiment, the gas sample Gp also flows through the tube 80 during the measurement.

A schematically shown actuator excites this flexural resonator to undamped oscillations. In the implementation shown, the actuator is implemented by two transducers 82.1, 82.2. Each transducer 82.1, 82.2 comprises a coil 83.1, 83.2 and a magnet 84.1, 84.2. In one mode, an alternating electrical voltage is applied to each transducer 82.1, 82.2, the transducer 82.1, 82.2 generates mechanical oscillations, and these mechanical oscillations are transmitted to the tube 80, preferably by a mechanical connection between the magnet 84.1, 84.2 and the tube 80.

The two legs 80.1, 80.2 span a plane. The two directions of the oscillations generated by the excitation are orthogonal to this plane. An area B of the tube 80 is set into oscillation. The actuator 84 is then switched off. Area B then continues to oscillate, namely at its natural frequency after a transient phase.

The area B of the tube 80 and the part of the gas sample Gp that is located inside this area B take part in the oscillation. This oscillating system has a mass, wherein this mass is the sum of the mass of area B and the mass of the gas sample part in area B. The mass of area B is predetermined by the configuration and remains constant. The volume that area B provides for the gas sample Gp also remains constant. As a rule, the mass of that part of the gas sample Gp that is located in area B can be regarded as constant during a measurement. The density p sought is then proportional to the mass of the gas sample part.

The two converters 82.1 and 82.2 are then switched off. As a result, the system just described oscillates at a natural frequency t. This natural frequency t depends on the mass of the system and is measured. In the implementation shown, the two converters 82.1, 82.2 function in a different mode after switching off and convert the mechanical oscillations of area B into an alternating voltage. The frequency of this alternating voltage is equal to the natural frequency t of area B. An amplifier 85 amplifies this alternating voltage. An evaluation unit 86 receives a signal from the amplifier 85 and supplies the required density p.

The sought density p is related to the measured natural frequency t as follows:

$$ρ = A * {τ}^{2} - B . \qquad ( 1 )$$

A and B are two constants of the density sensor (flexural resonator) 59. In a previous calibration, these two constants A and B are determined empirically, for example by means of a regression analysis. For this purpose, at least two different gases with known and differing densities are sent through the density sensor 59 and the respective resulting natural frequency is measured.

FIGS. 7A and 7B schematically show a flexural resonator 60 in the form of a quartz crystal (oscillating quartz). This quartz crystal has the shape of a tuning fork with two legs 61.1 and 61.2. FIG. 7A schematically shows this quartz crystal. Electrodes are connected to this flexural resonator 60 in such a way that two opposing fields occur in the x-direction. This forces a bending in the z-direction. FIG. 7B illustrates the oscillations of these two legs 61.1 and 61.2, wherein the x-direction and the z-direction lie in the plane of the drawing. A feedback loop ensures that the forced frequency is equal to the natural frequency t.

One such flexural resonator 60 is described, for example, in Th. Voglhuber-Brunnmeier et al: Fluid Sensing Using Quartz Tuning Forks-Measurement Technology and Applications, Sensors, Vol. 19 (2019) No. 10 (which is hereby incorporated by reference).

In one embodiment, the following context is used:

$$ρ = {[ - \frac{B}{2 A} + \sqrt{{( \frac{B}{2 A} )}^{2} + {( \frac{τ - C}{A} )}^{2}} ]}^{2} \qquad ( 2 )$$

Again, A, B and C are device-specific constants that are determined in advance by calibration. Because three constants have to be determined, at least three different gases with known and differing densities are used for calibration.

As already explained, the sensor arrangement 50 is intended to measure the respective concentration (proportion) in vol.-% of oxygen and anesthetic agent (anesthetic gas) in the tapped gas sample Gp. Other components may be present in this gas sample Gp, which can falsify this measurement without suitable countermeasures. In particular, the noble gas argon (Ar) in the gas sample Gp can falsify the measurement. It is well-known that the proportion of argon in the ambient air is approximately 0.93% by volume. However, the concentration of argon in the gas sample Gp can be significantly higher than that of argon in the ambient air. This situation occurs in particular if the concentration of oxygen in the ventilation circuit 40 and therefore in the gas sample Gp is greater than the concentration of oxygen in the ambient air, and in particular if the gas mixture Gg is generated from ambient air and the oxygen concentration has been increased with the aid of a so-called oxygen concentrator.

In some situations, information about the measured argon concentration is also used by another sensor or other component of the anesthesia device 1. For example, the measured argon concentration is compared with an upper limit. As soon as this upper limit for the argon concentration is reached, the ventilation circuit 40 is flushed.

In the embodiment example, the gas sample Gp contains n different components, including nitrogen (N₂), pure oxygen (O₂), carbon dioxide (CO₂), an anesthetic gas (A gas), nitrous oxide (N₂O), water (H₂O) and argon (Ar). The respective density ρ(xᵢ) (i=1, . . . , n) and the respective thermal conductivity λ(xᵢ) of these n components are known and are predetermined. Preferably, the sensor arrangement 50 has at least temporary read access to a data memory in which the densities and thermal conductivities are stored. The following table shows examples of the respective density and the respective thermal conductivity of components that occur or can occur in the ventilation circuit 40 and thus in the gas sample Gp, wherein the densities and the thermal conductivities relate to a reference ambient temperature of 15 degrees C. and a reference ambient pressure of 1 bar.

TABLE 1
Thermal conductivity and density of various gas components

| | Density ρ(xᵢ) | Thermal conductivity λ(xᵢ) |
|---|---|---|
| Gas xᵢ | [g/dm]³ | [W/mK] |
| Oxygen (O)₂ | 1.336 | 0.02615 |
| Nitrogen (N)₂ | 1.169 | 0.02566 |
| Carbon dioxide (CO)₂ | 1.8474 | 0.01643 |
| Dry ambient air | 1.209 | 0.02603 |
| Water vapor | 1 | 0.0199 |
| Isoflurane | 7.701 | 0.00942 |
| Nitrous oxide (N₂O) | 1.848 | 0.0173 |
| Argon (Ar) | 1.669 | 0.01782 |

The gas components have different densities. It can be seen that the anesthetic isoflurane mentioned as an example has a density several times greater than the other gas components.

As explained above, an optional pressure sensor 36 measures the pressure in the ventilation circuit 40 and preferably provides the airway pressure PAW. The optional pressure sensor 58 measures the ambient pressure P_{amb}, the optional temperature sensor 39 measures the ambient temperature Temp_{amb}, and an optional pressure sensor 57 measures the pressure P_{cell} of the gas sample Gp at the inlet of the sensor arrangement 50. The values listed in Table 1 refer to a specific reference ambient temperature and a specific reference ambient pressure. Optionally, the signal processing unit 30 uses signals from the sensors 36, 58 and/or 39 to derive the actual thermal conductivity and the actual density of the possible gas components at the actual ambient temperature Temp_{amb} and the actual ambient pressure P_{amb}.

The respective concentration ξ(xᵢ), i.e. the proportion in % by volume, of each component xᵢ (i=1, . . . , n) is sought.

In many cases, with sufficient accuracy it is valid that the density p of the gas sample Gp is a weighted average of the respective densities ρ(xᵢ) of the n components, i.e. the following applies:

$$ρ = ξ ( {x}_{1} ) * ρ ( {x}_{1} ) + … + ξ ( {x}_{n} ) * ρ ( {x}_{n} ) . \qquad ( 3 )$$

The sought concentrations ξ(x₁), . . . , ξ(xₙ) of the gas components x₁, . . . , xₙ act as the weight factors.

With reference to FIG. 2 to FIG. 5, a sensor 53 was described above that measures a detection variable that correlates with the thermal conductivity 2 of the gas sample Gp. In the example in FIG. 2, this is a detection variable of the thermocouple 8, in the example in FIG. it is the signal 76. In many cases, it is true with sufficient accuracy that the thermal conductivity λ of the gas sample Gp is a weighted average of the respective thermal conductivities λ(xᵢ) of the components:

$$λ = ξ ( {x}_{1} ) * λ ( {x}_{1} ) + … + ξ ( {x}_{n} ) * λ ( {x}_{n} ) . \qquad ( 4 )$$

The sought concentrations ξ(x₁), . . . , ξ(xₙ) of the gas components x₁, . . . , xₙ act as the weight factors.

It is also possible to use the following functional dependency:

$$λ = {\sum }_{i = 1}^{n} \frac{λ ( {x}_{i} )}{1 + {\sum }_{\begin{matrix}k = 1 \\ k \# i\end{matrix}}^{n} G ( i , k ) \frac{ξ ( {x}_{k} )}{ξ ( {x}_{i} )}} \qquad ( 5 )$$

The factors G(i,k) (i=1, . . . , n; k=1, . . . , n) can be determined empirically in advance.

The sensor 53 is also able to measure the magnetically modulated thermal conductivity λ_{2f} of the gas sample Gp, for example by using the periodic component of the detection variable of the embodiment according to FIG. 2 or by using the oscillating signal 75 of the circuit of FIG. 5. Because O₂ is a paramagnetic gas, λ_{2f} significantly depends on the O₂ concentration ξ(O₂). In general, the following functional dependence f can be applied:

$${λ}_{2 f} = {f [ ξ ( {O}_{2} ) , ξ ( {N}_{2} O ) , ξ ( {CO}_{2} ) , ξ ( {H}_{2} O ) , ξ ( Ar ) , {Temp}_{amb} , P ]}_{cell} \qquad ( 6 )$$

The functional dependency f is predetermined. Temp_{amb} denotes the ambient temperature, which is measured by the temperature sensor 39, for example. As a rule, the temperature of the gas sample Gp does not differ significantly from the ambient temperature Temp_{amb}. The O₂ concentration is approximately linear. The pressure P_{cell} is measured by sensor 57, for example.

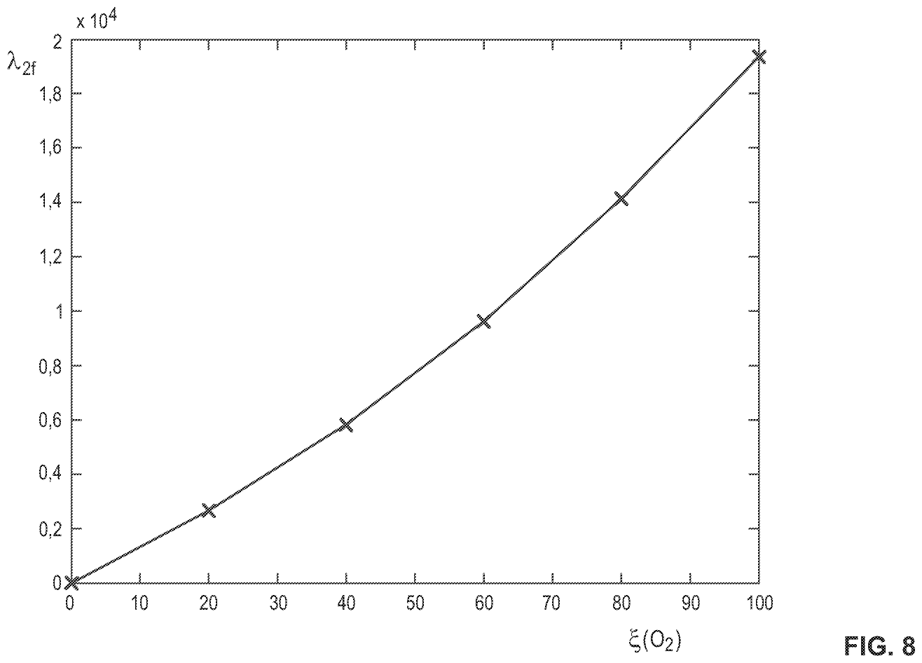

FIG. 8 shows an example of the magnetically modulated thermal conductivity λ_{2f} of a gas mixture as a function of the O₂ concentration ξ(O₂). In this case, the gas mixture consists only of oxygen and N₂O. The concentration ξ(O₂) of oxygen in the gas mixture in vol.-% is plotted on the x-axis, and the magnetically modulated thermal conductivity λ_{2f} is plotted on the y-axis. It can be seen that the relationship λ_{2f}=λ_{2f}[ξ(O₂)] is approximately linear, but not ideally linear.

The sensor 54 measures the CO₂ concentration ξ(CO₂) and the N₂O concentration ξ(N₂O). The pressure sensor 57 measures the pressure P_{cell}. A capacitive sensor (not shown) measures the H₂O concentration ξ(H₂O), i.e. the concentration of water vapor in the gas sample Gp. The density sensor 59, 60, which was described above with reference to FIG. 6 and FIG. 7, measures the density ρ of the gas sample Gp.

The CO₂ concentration ξ(CO₂), the N₂O concentration ξ(N₂O) and the water vapor concentration ξ(H₂O) are measured directly. This leaves three unknown components, namely the O₂ concentration ξ(O₂), the anesthetic concentration ξ(A gas) and the argon concentration ξ(Ar). To determine these three unknowns, the three equations (3), (4) and (6) are used. Taken together, a system of equations with three unknowns, namely ξ(O₂), ξ(A gas) and ξ(Ar), and three equations, namely (3), (4) and (6), is used.

In one implementation, three values ξ(x₁), ξ(x₂) and ξ(x₃) are calculated for the three unknowns ξ(O₂), ξ(A-gas) and ξ(Ar) in such a way that a target function Z with

$$Z [ ξ ( x 1 ) , ξ ( x 2 ) , ξ ( x 3 ) ] = {α}_{1} * {\{ {ρ}_{meas} - ρ [ ξ ( {x}_{1} ) , ξ ( {x}_{2} ) , ξ ( x 3 ) ] \}}^{2} + {α}_{2} * \{ {λ}_{meas} - λ [ ξ ( {x}_{1} ) , ξ ( {x}_{2} ) , ξ ( {x}_{3} ) ] \} {+}^{2} {α}_{3} * {\{ {λ}_{, 2 fmeas} - {λ}_{2 f} [ ξ ( {x}_{1} ) , ξ ( {x}_{2} ) , ξ ( {x}_{3} ) ] \}}^{2} \qquad ( 7 )$$

is minimized. Here, ρₘₑₐₛ denotes the measured density, λₘₑₐₛ the measured thermal conductivity and λ,_{2fmeas} the measured magnetically modulated thermal conductivity. ρ[ξ(x₁), ξ(x₂), ξ(x₃)] denotes the density that occurs according to equation (3), λ[ξ(x₁), ξ(x₂), ξ(x₃)] is the thermal conductivity according to equation (4) or (5) and λ_{2f} [ξ(x₁), ξ(x₂), ξ(x₃)] is the magnetically modulated thermal conductivity according to equation (6). The three factors α₁, α₂, α₃ are selected so that all three summands of the target function Z have the same order of magnitude and/or different accuracy requirements for the measurement of the three concentrations ξ(x₁), ξ(x₂), ξ(x₃) are met. The sum of the three factors α1, α2, α3 is 1.

Obviously, the sum of the gas component parts is 1, so the following applies

$$ξ ( {x}_{1} ) + … + ξ ( {x}_{n} ) = 1. \qquad ( 8 )$$

Equation (8) can be used to derive the N₂ concentration ξ(N₂).

While specific embodiments of the invention have been shown and described in detail to illustrate the application of the principles of the invention, it will be understood that the invention may be embodied otherwise without departing from such principles.

### LIST OF REFERENCE CHARACTERS

| | |
|---|---|
| 1 | Anesthesia machine, belongs to the ventilation arrangement |
| | 200 |
| 2 | Measuring chamber of the sensor 53, formed in the air gap 3 |
| 3 | Air gap, provides the measuring chamber 2 |
| 4, 5 | Field coils, generate together with the pole pieces 6, 7 a |
| | magnetic field in the measuring chamber 2 |
| 6, 7 | Pole pieces, generate together with the field coils 4, 5 a |
| | magnetic field in the measuring chamber 2 |
| 8 | Thermocouple, comprises the wires 17, 18 and the support |
| | wires 15, 16 |
| 9 | Wall of measuring chamber 2 |
| 10 | Inlet into the measuring chamber 2 |
| 11 | Outlet from measuring chamber 2 |
| 12 | Connection point between the support wire 15 and the wire 17 |
| 13 | Connection point between wires 17 and 18 |
| 14 | Connection point between the support wire 16 and the wire 18 |
| 15, 16 | Support wires for the thermocouple 8 |
| 17, 18 | Wires of the thermocouple 8 are connected to each other at the |
| | junction 13 |
| 19 | Measuring resistance of the thermocouple 8 |
| 20 | Voltage source |
| 21 | Patient-side coupling unit in the form of a mouthpiece or |
| | breathing mask, connected to the Y-piece 22 |
| 22 | Y-piece, which connects the patient-side coupling unit 21 with |
| | the inspiration line 32 for the supply (inhalation, inspiration) |
| | of the breathable gas mixture Gg and the expiration line 33 for |
| | the removal (exhalation, expiration) of the exhaled gas |
| | mixture At |
| 23a | Non-return valve that allows a gas flow in the direction of the |
| | patient P in the inspiration line 32 and blocks it in the opposite |
| | direction |
| 23b | Non-return valve that allows a gas flow away from patient P |
| | in the expiratory line 33 and blocks it in the direction of |
| | patient P |
| 24a | Fluid delivery unit in the form of a blower, which generates a |
| | volume flow of the breathable gas mixture Gg in the direction |
| | of the patient P |
| 24b | Controllable valve that generates the ventilation breaths |
| 24c | PEEP valve, which maintains an end-expiratory pressure in |
| | the patient's lungs P |
| 25a | Carbon dioxide absorber, absorbs carbon dioxide from the |
| | ventilation circuit 40 |
| 26 | Breathing bag via which the ventilation circuit 40 can be |
| | driven |
| 27 | Fluid flow of fresh air or other fresh gas to the ventilation |
| | circuit 40 |
| 28 | Fluid flow of vaporous anesthetic to the ventilation circuit 40, |
| | generated by the anesthetic dispenser 31 |
| 29 | Adjustable pressure relief valve that can release gas from the |
| | ventilation circuit 40 |
| 30 | Signal processing unit for the sensor arrangement 50, |
| | evaluates signals from the sensors 53, 54, 57 and 58 |
| 31 | Anesthetic dispenser, generates the anesthetic flow 28 |
| 32 | Inspiratory line for inhalation, connected to the Y-piece 22, |
| | has the non-return valve 23a |
| 33 | Expiratory line for exhalation, connected to the Y-piece 22, |
| | has the non-return valve 23b |
| 34 | Branch point of the sampling tube 52, located near the |
| | Y-piece 22 |
| 35 | Ventilation control unit, controls the fluid delivery unit 24a, |
| | the valve 24b and the anesthetic dispenser 31, receives signals |
| | about the respective gas concentration and messages from the |
| | signal processing unit 30 |
| 36 | Pressure sensor in the ventilation circuit 40, preferably |
| | measures the pressure P_{aw} applied to the patient P |
| 37 | Junction point at which the discharge tube 56 joins the |
| | ventilation circuit 40 is located upstream of the carbon dioxide |
| | absorber 25a |
| 39 | Temperature sensor, measures the temperature Temp_{amb} in the |
| | vicinity of the ventilator 1 |
| 40 | Ventilation circuit, through which the patient P is artificially |
| | ventilated, comprises the patient-side coupling unit 21, the |
| | Y-piece 22, the inspiratory line 32 and the expiratory line 33 |
| 42 | Power amplifier between the voltage source 43 and the field |
| | coil 5 |
| 43 | Voltage source, emits a sinusoidal electrical voltage |
| 44 | Optional display device of the sensor 53 |
| 50 | Sensor arrangement, comprising the sensors 53, 54, 57, the |
| | pressure sensor 59, 60 and the pump 55 |
| 51 | Water trap upstream of the sensor arrangement 50 |
| 52 | The sampling tube, through which the gas sample Gp is taken |
| | from the ventilation circuit 40, starts at a branch point 34 |
| | between the patient-side coupling unit 21 and the Y-piece 22 |
| | and leads to the water trap 51 |
| 53 | Sensor for the concentration of oxygen |
| 54 | Sensor for the concentration of CO₂, H₂O and anesthetic in |
| | the gas sample Gp |
| 55 | Pump which sucks a gas sample Gp into the sampling tube 52 |
| 56 | Discharge tube, connects the sensor arrangement 50 with the |
| | ventilation circuit 40 |
| 57 | Pressure sensor of the sensor arrangement 50, measures the |
| | pressure of the gas sample Gp |
| 58 | Sensor for ambient pressure P_{amb} |
| 59 | Mechanical flexural resonator measuring the density of the gas |
| | sample Gp comprises the housing 83, the tube 80 with the legs |
| | 80.1, 80.2, the attachment point 81, the electromagnetic |
| | transducer 82.1, 82.2, the actuator 84, the amplifier 85 and the |
| | evaluation unit 86 |
| 60 | Electronic flexural resonator in the form of a quartz crystal, |
| | comprises the legs 61.1, 61.2 |
| 62 | Electromagnet, generates a time-varying magnetic field in the |
| | air gap 3, comprises the coil 63 |
| 63 | Electric coil of the electromagnet 62 |
| 64 | The measuring unit, implemented as a chip, comprises the |
| | heat conduction measuring element 65, the heating unit 66 and |
| | the membrane 67 |
| 65 | Electrically controllable heat conduction measuring element of |
| | measuring unit 64 |
| 66 | Electrically controllable heating unit of measuring unit 64 |
| 67 | Diaphragm of the measuring unit 64 |
| 68 | Electrically conductive connecting element between the two |
| | heating elements of the heating unit 66 |
| 69 | Frame of the measuring unit 64 |
| 70 | Amplifier |
| 71 | Voltage divider |
| 72 | DC voltage source |
| 73 | Low-pass filter, supplies the signal 76 |
| 74 | High-pass filter, supplies the signal 75 |
| 75 | Signal at the output of the high-pass filter 74, correlates with |
| | the concentration ξ(O₂) of oxygen |
| 76 | Signal at the output of the low-pass filter 73, correlated with |
| | the thermal conductivity λ of the gas sample Gp |
| 77 | Measuring position of the measuring unit 64 |
| 80 | U-shaped tube, has the two legs 80.1, 80.2, holds the gas |
| | sample Gp, is excited to vibrate |
| 80.1, 80.2 | Leg of the U-shaped tube 80 |
| 81 | Fastening point at which the two legs 80.1, 80.2 are clamped |
| 82.1, 82.2 | Electromagnetic converters, convert electrical AC voltage into |
| | mechanical vibrations in one mode and mechanical vibrations |
| | into electrical AC voltage in another mode, comprise the |
| | coils 83.1, 83.2 and the magnets 84.1, 84.2 |
| 83.1, 83.2 | Coil of the converter 82.1, 82.2 |
| 84.1, 84.2 | Magnet of the converter 82.1, 82.2 |
| 85 | Amplifier of the density sensor 59 |
| 86 | Evaluation unit of the density sensor 59 |
| 83 | Housing of the seal sensor 59, accommodates the tube 80 |
| 100 | Measurement system, comprises the sensor arrangement 50, |
| | the water trap 51, the signal processing unit 30 and the tubes |
| | 52 and 56 |
| 200 | Ventilation arrangement, comprises the anesthesia machine 1, |
| | the measurement system 100, the patient-side coupling unit 21 |
| | and the lines 32 and 33 |
| At | Gas mixture exhaled by the patient P, contains carbon dioxide |
| | and an anesthetic agent |
| G(i, k) | Empirically determined weight factors |
| Gg | Breathable gas mixture, which the ventilator 1 delivers to the |
| | patient-side coupling unit 21, contains oxygen, an anesthetic |
| | agent and argon |
| Gp | Gas sample, is branched off from the ventilation circuit 40, |
| | passed through the sampling tube 52 to the sensor |
| | arrangement 50 and fed back into the ventilation circuit 40 |
| | through the discharge tube 56 |
| Λ | Thermal conductivity of the gas sample Gp |
| λ(x)ᵢ | Thermal conductivity of the gas component xᵢ (i = 1, . . . , n) |
| | of the gas sample Gp |
| λ_{2f} | Magnetically modulated thermal conductivity, is an indicator |
| | of the oxygen concentration in the gas sample Gp |
| λ₂ f(x)ᵢ | Magnetically modulated thermal conductivity of the gas |
| | component xᵢ (i = 1, . . . , n) of the gas sample Gp |
| N | Number of gas components in the gas sample Gp |
| P | Patient who is artificially ventilated and connected to the |
| | patient-side coupling unit 21 |
| P_{amb} | Pressure in the vicinity of the ventilation assembly 200, |
| | measured by the pressure sensor 58 |
| P_{aw} | Airway pressure in the patient-side coupling unit 21, is |
| | measured by the pressure sensor 58 in the inspiratory line 32 |
| P_{cell} | Pressure of the gas sample Gp at the inlet of the sensor |
| | arrangement 50, measured by the pressure sensor 57 |
| P | Density of the gas sample Gp |
| ρ(x)ᵢ | Density of the gas component xᵢ (i = 1, . . . , n) of the gas |
| | sample Gp |
| Temp_{amb} | Ambient temperature, measured by temperature sensor 39 |
| ξ(x)ᵢ | Concentration in vol.-% of the gas component xᵢ (i = |
| | 1, . . . , n) of the gas sample Gp |
| x₁, . . . , | Gas components of the gas sample Gp |
| xₙ | |

What is claimed is:

1. A sensor arrangement for measuring a respective concentration of three gas components of a gas sample, wherein a respective density and a respective thermal conductivity of each of the three gas components is predetermined, wherein one of the three gas components is a paramagnetic gas, the sensor arrangement comprising:
a measuring chamber configured to receive a gas sample;
a thermal conductivity sensor configured to measure a thermal conductivity of the gas sample; and
a density sensor configured to measure a density of the gas sample;
wherein the sensor arrangement is configured to feed or guide the gas sample into the measuring chamber, to apply a magnetic field with an oscillating magnetic field strength to the measuring chamber and to measure a magnetically modulated thermal conductivity of the gas sample in the measuring chamber,
wherein the magnetically modulated thermal conductivity is a proportion of the thermal conductivity of the gas sample that oscillates depending on the magnetic field strength, and
wherein the sensor arrangement is configured to determine the three gas component concentrations using the predetermined densities of the three gas components, the predetermined thermal conductivities of the gas components, the measured thermal conductivity of the gas sample, the measured magnetically modulated thermal conductivity of the gas sample, and the measured density of the gas sample.

2. A sensor arrangement according to claim 1, further comprising
a heating unit configured to supply thermal energy to a gas sample in the measuring chamber,
wherein the sensor arrangement is configured to measure an electrical detection variable which variable correlates with the thermal conductivity of the gas sample, and, by filtering the electrical detection variable, to derive an oscillating signal, which signal oscillates as a function of the magnetic field strength, and a further signal, the time course of which does not depend on the oscillating magnetic field strength, and
wherein the sensor arrangement is configured to measure the magnetically modulated thermal conductivity of the gas sample as a function of the oscillating signal and to measure the thermal conductivity of the gas sample as a function of the further signal.

3. A sensor arrangement according to claim 1,
wherein the density sensor comprises a flexural resonator, the flexural resonator comprising an oscillating body,
wherein the sensor arrangement is configured to guide the gas sample through or along the oscillating body,
wherein the flexural resonator is configured to cause the oscillating body to vibrate and to measure the natural frequency of the vibrating oscillating body, and
wherein the sensor arrangement is configured to determine the density of the gas sample as a function of the measured natural frequency of the oscillating body.

4. A sensor arrangement according to claim 1, in combination to form a ventilation arrangement for artificial ventilation of a patient, the combination further comprising:
a medical device;
a patient-side coupling unit; and
a ventilation control unit to form a ventilation arrangement for artificial ventilation of a patient,
wherein the patient-side coupling unit is configured to be connected to the patient,
wherein the ventilation arrangement is configured to convey a breathable gas mixture from the medical device to the patient-side coupling unit, the gas mixture comprising the three gas components,
wherein a target range is predetermined for the concentration of one gas component of the three gas components in the gas mixture,
wherein the ventilation arrangement is configured to branch off a gas sample from the gas mixture which is conveyed to the patient-side coupling unit, and to direct the branched-off gas sample to the sensor arrangement, and
wherein the sensor arrangement is configured to measure an actual concentration of the gas component in the branched-off gas sample for which the target range is given.

5. A sensor arrangement combination forming the ventilation arrangement according to claim 4,
wherein the ventilation control unit is configured to perform a closed-loop control of the concentration of the gas component for which the target range is given in the gas mixture and to use the measured gas component concentration for the closed-loop control, and
wherein a control objective is to ensure that the actual gas component concentration in the gas mixture remains within a predetermined setpoint range.

6. A measuring process for measuring a respective concentration of three gas components of a gas sample, wherein a respective density and a respective thermal conductivity of each one of the three gas components are predetermined and wherein one of the three gas components is a paramagnetic gas, the measuring process comprising the steps of:
measuring a thermal conductivity of the gas sample;
measuring a density of the gas sample;
feeding or guiding the gas sample into a measuring chamber;
applying a magnetic field with an oscillating magnetic field strength to the gas sample in the measuring chamber;
measuring a magnetically modulated thermal conductivity of the gas sample in the measuring chamber, wherein the magnetically modulated thermal conductivity is a proportion of the thermal conductivity of the gas sample that oscillates as a function of the magnetic field strength; and
determining the three gas component concentrations based on the predetermined densities of the gas components, the predetermined thermal conductivities of the gas components, the measured thermal conductivity of the gas sample, the measured magnetically modulated thermal conductivity of the gas sample, and the measured density of the gas sample.

7. A measuring process according to claim 6, wherein, as the thermal conductivity, the thermal conductivity of the gas sample in the measuring chamber is measured, and the measuring process further comprises:
heating a heating unit to supply thermal energy to the gas sample in the measuring chamber;
measuring an electrical detection variable of the heated heating unit, wherein the detection variable correlates with the thermal conductivity of the gas sample; and
by filtering the electrical detection variable, deriving an oscillating signal, which oscillates as a function of the magnetic field strength, and a further signal, a time course of which does not depend on the oscillating magnetic field strength,
wherein the magnetically modulated thermal conductivity of the gas sample is measured as a function of the oscillating signal, and the thermal conductivity of the gas sample is measured as a function of the other signal.

8. A measuring process according to claim 6, further comprising:
passing the gas sample through or along an oscillating body of a flexural resonator,
setting the oscillating body into oscillation; and
measuring a natural frequency of the oscillating body,
wherein the density of the gas sample is determined as a function of the measured natural frequency of the oscillating body.

9. A measuring process according to claim 6,
wherein the thermal conductivity, the magnetically modulated thermal conductivity and/or the density of the gas sample additionally depends on a concentration of a fourth gas component of the gas sample,
wherein the density of the fourth gas component is predetermined, and
wherein the measuring process further comprises measuring or otherwise determining the concentration of the fourth gas component, and
wherein in the step of determining the three gas component concentrations, the measured concentration of the fourth gas component is additionally used.

10. A measuring process according to claim 9,
wherein the step of measuring the concentration of the fourth gas component comprises the steps of
emitting, with a radiation source, electromagnetic radiation or sound such that at least a part of the radiation penetrates the gas sample and impinges on a detector, and
with the detector, measuring an intensity of the impinging electromagnetic radiation or the impinging sound and generating a signal depending on the measured intensity,
wherein the concentration of the fourth gas component is measured as a function of the signal generated by the detector, which signal indicates the concentration of the fourth gas component.

11. A measuring process according to claim 6,
wherein three functional relationships, three difference indicators and one target function are predetermined,
wherein a first functional relationship of the three functional relationships describes thermal conductivity of the gas sample as a function of the three gas component concentrations,
wherein a second functional relationship of the three functional relationships describes magnetically modulated thermal conductivity of the gas sample as a function of the three gas component concentrations,
wherein a third functional relationship of the three functional relationships describes density of the gas sample as a function of the three gas component concentrations,
wherein a first difference indicator of the three difference indicators describes an indictor of a difference between the measured thermal conductivity of the gas sample and the thermal conductivity derived using the first functional relationship,
wherein a second difference indicator of the three difference indicators describes an indicator of a difference between the measured magnetically modulated thermal conductivity of the gas sample and the magnetically modulated thermal conductivity derived using the second functional relationship,
wherein a third difference indicator of the three difference indicators describes an indicator of a difference between the measured density of the gas sample and the density derived using the third functional relationship,
wherein the target function is an aggregation of the three difference indicators, and
wherein the measuring process further comprises:
inserting the three measured values for the thermal conductivity, the magnetically modulated thermal conductivity and the gas sample density in the target function; and
calculating a value triplet for the three gas component concentrations such that the target function is minimized.

12. A measuring process according to claim 6, wherein the three gas components whose concentrations are measured in the branched gas sample are oxygen, anesthetic and argon.

13. A ventilation process for artificial ventilation of a patient, wherein the patient is connected to a patient-side coupling unit and wherein the ventilation process comprises the steps of:
conveying a breathable gas mixture from a medical device to the patient-side coupling unit,
wherein a target range for the concentration of a gas component in the gas mixture is predetermined,
branching off a gas sample from the gas mixture;
measuring an actual concentration of the gas component for which the target range is predetermined with the measuring process according to claim 6, wherein the gas component for which a target range is predetermined is one of the three gas components whose concentrations are measured; and
by closed-loop control, controlling the actual concentration of the gas component in the gas mixture with the control objective that the actual oxygen concentration of the gas component for which the target range is predetermined is within the predetermined target range, wherein the measured actual concentration of the gas component for which the target range is predetermined is used for the closed-loop control.

14. The ventilation process according to claim 13,
wherein the gas mixture is generated using ambient air,
wherein the oxygen concentration is increased during the generation of the gas mixture, and
wherein the three gas components which concentrations are measured in the branched gas sample are oxygen, an anesthetic and argon.

15. The ventilation process according to claim 13, wherein the gas mixture is conveyed through a fluid guide unit from the medical device to the patient-side coupling unit, and
Wherein the detection of the event that at least one gas component in the conveyed gas mixture is outside the target range predetermined for this gas component triggers the step of flushing the fluid guide unit.
